(19) United States
(12) Patent Application Publication
(10) Pub. No.: US 2026/0115391 A1
(43) Pub. Date: Apr. 30, 2026

(54) NEBULIZER

(71) Applicant: Stamford Devices Limited, Galway (IE)

(72) Inventors: Michael CASEY, Cornamona (IE); Joseph GREHAN, Gort (IE); Kieran HYLAND, Galway (IE); John POWER, Moycullen (IE)

(73) Assignee: Stamford Devices Limited, Galway (IE)

(21) Appl. No.: 19/431,298

(22) Filed: Dec. 23, 2025

Related U.S. Application Data
(63) Continuation of application No. 17/307,057, filed on May 4, 2021, now Pat. No. 12,527,923.
Application No. 17/307,057 is a continuation of application No. 16/019,026, filed on Jun. 26, 2018, now Pat. No. 11,027,076.
Application No. 16/019,026 is a continuation of application No. 13/870,376, filed on Apr. 25, 2013, now Pat. No. 10,029,053.
(60) Provisional application No. 61/642,284, filed on May 3, 2012.

(51) Int. Cl. A61M 11/00; B05B 12/00; B05B 17/00

(52) U.S. Cl. CPC A61M 11/00; A61M 11/005; B05B 12/00; B05B 17/0646; A61M 2205/502; A61M 2205/52; A61M 2205/587; A61M 2205/70

(57) ABSTRACT

A nebulizer comprises a controller linked at its output to a nebulizer head, and at its input to a USB cable and USB plug for connection to a host system. The link between the USB plug and the controller is a USB cable with power and data channels. The controller comprises a boost circuit, a microcontroller 11, and a drive circuit. The latter provides power and control signals via a cable and proprietary plug to the nebulizer head. These signals provide power and control for a vibrating membrane receiving a liquid to be aerosolised. The controller has a housing with LED status lamps, and an ON/OFF button. The controller can be controlled via a host, either locally or remotely.

### CROSS REFERENCE TO RELATED APPLICATIONS

The present application claims the benefit of U.S. provisional application Ser. No. 61/642,284, filed on May 3, 2012, the contents of which are herein incorporated by reference in their entirety.

### FIELD OF THE INVENTION

The invention relates to a nebulizer, and more particularly to power supply and control of aerosol generation in nebulizers.

### PRIOR ART DISCUSSION

Aerosol delivery systems can aerosolise a broad range of liquids across a particle range 1-50 μm MMAD. Such systems have an aperture plate with holes of pre-determined size and which is coupled with a vibrational mechanism powered by a piezo. The aperture plate is vibrated at a frequency of typically 120 to 150 kHz and this action causes the liquid to break surface tension and it creates an aerosol plume as droplets pass through the aperture plate. An example is described in WO2010035252 (Stamford Devices Ltd).

WO2012/026963 (Rubin) describes an aerosol delivery system having an integral USB plug.

### SUMMARY OF THE INVENTION

According to the invention, there is provided a nebulizer comprising a nebulizer head for generating an aerosol, a controller for delivering power and control signals to the nebulizer head, and a standard universal bus for delivering power and control signals to the controller,
wherein the standard universal bus comprises a universal plug and a universal cable extending from said plug to the controller,
wherein the controller is linked by a cable and a plug to the nebulizer head; and
wherein the controller comprises a voltage boost circuit linked at its input with the universal bus, a processor, and a drive circuit for delivering power to the nebulizer head under control of the processor.

In one embodiment, the universal bus is a USB bus, and wherein total power requirement of the controller and of the nebulizer head falls within the power specification of the standard USB protocol.

In one embodiment, the controller provides to the nebulizer head no more than 500 mA at nominal 5V.

In one embodiment, the drive circuit is adapted to generate a waveform in the range of 120 kHz and 150 kHz.

In one embodiment, the boost circuit is adapted to generate a voltage supply for the processor and the drive circuit. Preferably, the voltage level is 12 V.

In one embodiment, the processor is adapted to upload the following information to a host via the universal bus:
power consumption, and/or
wet/dry state of the nebulizer head, and/or
nebulizer disconnect status, and/or
cable disconnect status, and/or
error or fault states, and/or
nebulization duration and time of nebulization.

In another embodiment, the controller is adapted to receive the following signals from a host via the universal bus and to use them to generate nebulizer head drive signals:
nebulization start/stop, and/or
nebulization time, and/or
nebulization flow rate, and/or
nebulization pulse rate, and/or
inspiratory/expiratory signal to enable phased nebulization

In another aspect, the invention provides a nebulizer system comprising a nebulizer as defined above in any embodiment, and a host system adapted to provide control signals to the controller via said bus.

In one embodiment, the host system is adapted to provide the control signals according to a patient dosing regime.

In one embodiment, the host system includes a component adapted to communicate with a remote server for download of control instructions and/or upload of nebulizer data.

In one embodiment, the host system includes a portable device such as a smartphone or tablet computer.

### DETAILED DESCRIPTION OF THE INVENTION

The invention will be more clearly understood from the following description of some embodiments thereof, given by way of example only with reference to the accompanying drawings in which:

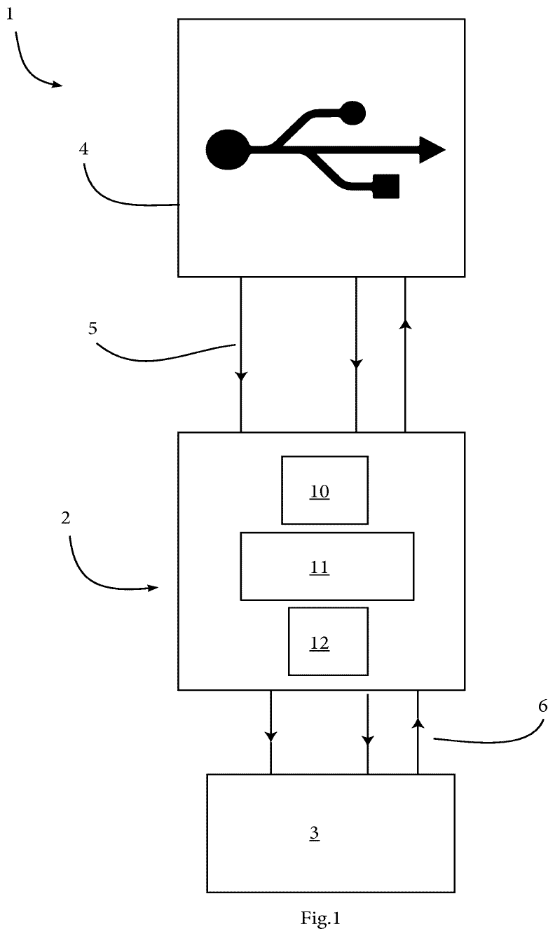

FIG. 1 is a block diagram illustrating architecture of a nebulizer of the invention; and

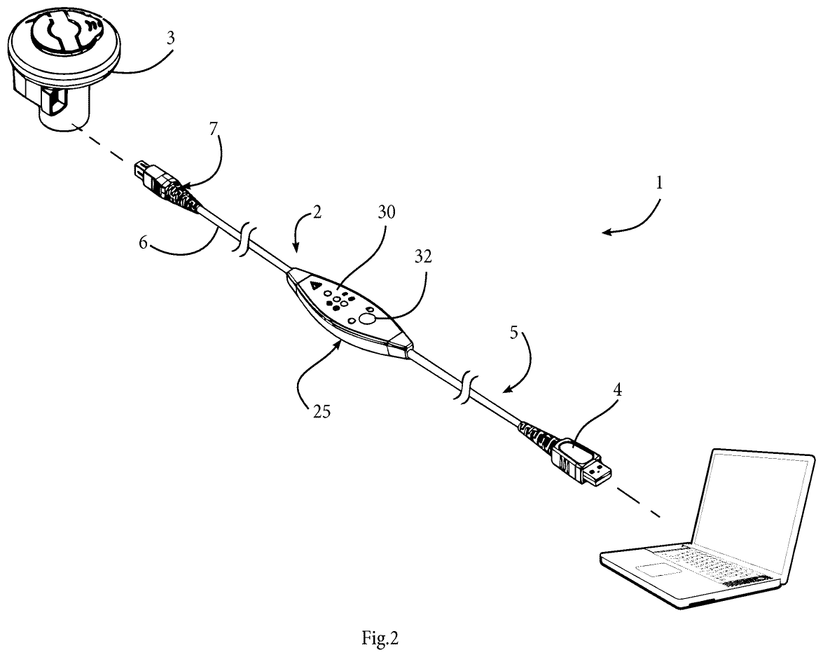

FIG. 2 is a perspective view of the nebulizer.

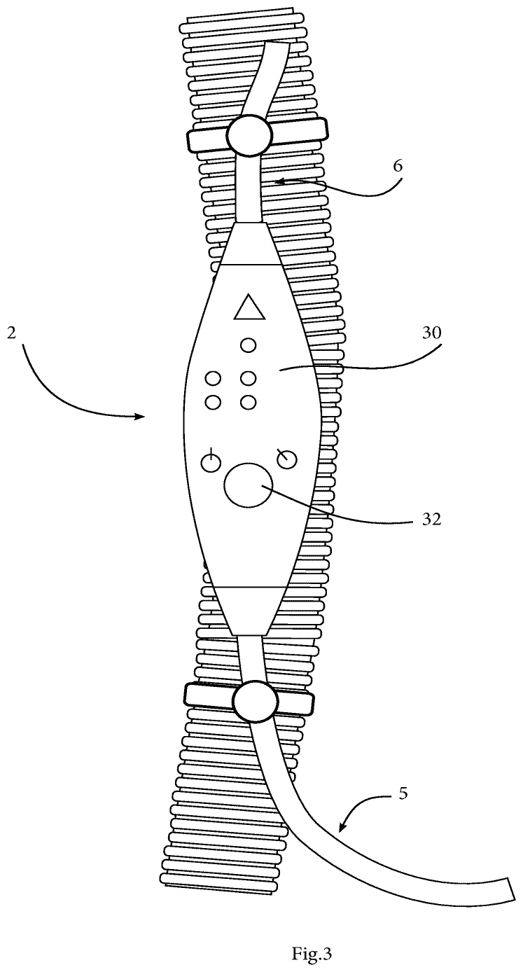

FIG. 3 shows the nebuliser in use in one example with the controller attached to a ventilation tube.

### DESCRIPTION OF THE EMBODIMENTS

Referring to the drawings, a nebulizer 1 comprises a controller 2 linked to a vibrating mesh nebulizer head 3. The nebulizer 1 also comprises a USB plug 4 for connection to a host system or device.

The link between the USB plug 4 and the controller 2 is a USB (Universal Serial Bus) cable 5 with power and data channels. The link between the controller 2 and the nebulizer head 3 is a proprietary cable 6 with power and data channels and a proprietary plug 7.

The controller 2 comprises a boost circuit 10 consisting of a custom optimized high frequency, high efficiency DC to DC converter with an integrated power switch capable of providing an output voltage and current profile suitable to drive the nebulizer load, There is also a drive circuit 12 utilizing a series inductor to generate the alternating AC voltage. The drive circuit 12 incorporates a high speed MOSFET driver controlled by a pulse width modulated signal from the microcontroller. There is also a microcontroller 11 with an integrated peripheral module featuring a full speed USB 2.0 compliant interface that can automatically change clock sources and power levels upon connection to a host. The latter provides power and control signals via the proprietary cable 6 and the proprietary plug 7 to the nebulizer head 3. These signals provide power and control for a vibrating membrane receiving a liquid to be aerosolised from a feed container. An example of the nebulizer head is described in our previous PCT application WO2012/046220. The controller 2 and the nebulizer head 3 require no more than 500 mA at nominal 5V to generate a desired aerosol.

The controller 2 has a housing 25 with LED status lamps 30, and an ON/OFF button 32. Communication takes place between the controller 2 and the USB plug 4 in compliance with the USB protocol.

The nebulizer drive circuit 12 consists of components to generate an output sine waveform of approximately 100V AC which is fed to the nebulizer head 3, causing aerosol to be generated. It uses inputs from the microcontroller 11 and the boost circuit 10 to achieve its output. The drive circuit 12 is matched to the impedance of a piezo ceramic element which causes the membrane to vibrate to ensure good energy transfer.

The microcontroller 11 generates a square waveform of 120 to 150 KHz which is sent to the drive circuit 12. The boost circuit 10 generates a nominal 12V DC voltage required by the drive circuit 12 from an input within the range of 4.75V to 5.25 V DC as per USB 2.0 electrical input requirements (released April 2000). The circuit is matched to the impedance of the piezo ceramic element within the nebulizer head 3 to ensure enhanced energy transfer. A drive frequency of 120 to 150 kHz is generated to drive the nebulizer head 3 membrane at close to its resonant frequency so that enough amplitude is generated to break off droplets and produce the aerosol. If this frequency is chopped at a lower frequency such that aerosol is generated for a short time and then stopped for a short time this gives good control of the nebulizer's flow rate. This lower frequency is called the “pulse rate”.

The drive frequency may be started and stopped as required using the microcontroller 11. This allows for control of flow rate by driving the nebulizer head 3 for any required pulse rate. The microcontroller 11 may control the ON and OFF times to an accuracy of milliseconds.

The nebulizer head 3 may be calibrated at a certain pulse rate by measuring how long it takes to deliver a known quantity of solution. There is a linear relationship between the pulse rate and the nebulizer flow rate. This may allow for accurate control over the delivery rate of the aqueous solution.

Because of use of the universal bus, in this case the USB cable 5 and the USB plug 4, the controller 2 can achieve very wide-ranging control of the nebulizer head 3. Also, it allows the controller 2 to be connected to a host having data processing and USB communication capability (such as a host computer or a portable device) for upload of information previously captured from the nebulizer head 3, or for download of configuration settings or other data to the controller 2. In combination, the host and the nebulizer 1 form a system which may advantageously be used, for example, for clinical trials or controlled hospital or home treatment regimes.

The controller 2 can upload in various embodiments the following nebulizer characteristics to the host:
power consumption, and/or
wet/dry state, and/or
nebulizer disconnect status, and/or
cable disconnect status, and/or
error or fault states, and/or
nebulization duration and time of nebulization.

The host can in various embodiments provide the following instructions to the controller 2:
nebulization start/stop, and/or
nebulization time, and/or
nebulization flow rate, and/or
nebulization pulse rate, and/or
inspiratory/expiratory signal to enable phased nebulization.

This will allow the controller 2 to be controlled via a host, either locally or remotely. The controller 2 may operate as a slave device, with the dosing regime determined by the host. This allows comprehensive control and treatment monitoring for a wide variety of situations such as in the home or in hospitals. If the controller is in communication with an external device, it can then act as a slave device and take commands form the external device. If it is powered by an external device, the mode of operation will be determined by the user input via the ON/OFF (power) button 32 and in this case the controller can be thought of being in “master” mode.

In other embodiments, the controller may be in the form of a hand held device, and may in fact be a mobile phone programmed to generate a user interface for nebulizer control. A specific mobile phone application could be generated to enable control of nebulization.

The arrangement of having the USB plug 4, the USB cable 5, the small hand-held controller 2, the proprietary cable 6 and the proprietary plug 7 allows convenience and versatility in use. For example, as illustrated in FIG. 3 the cables 5 and 6 along with the controller 2 may be secured by ties to a ventilator tube. However, they may be attached to any other tubular or elongate member. This allows convenience of access by care-givers and patients alike.

It will also be appreciated that the nebuliser can be plugged into any host system. The arrangement gives flexibility in a care-giving setting where there are multiple ventilators and patents. The USB interface allows link-up with a host device to allow remote control by a care-giver, for example, for control over the Internet to a host laptop computer into which the nebuliser is plugged.

Further, the extent of allowable local control may be limited to on/off control by limiting scope of the interface on the controller 2, the dosing control (pulse rate, flow rate, frequency etc.) being controlled from a remote location via a the host system. The controller may execute a program such as an “app” which allows a patient-specific dosing regime to be downloaded from to a mobile/tablet/PDA. The local program will then implement this new dosing regime.

There is excellent versatility because the nebuliser can be used with a ventilator, car socket, laptop computer, desktop computer, or battery pack. Because the controller is essentially part of the cabling it may not be by-passed accidentally.

The following are other benefits which arise from the invention in various embodiments:
Increased portability due to elimination of need for additional accessories, such as battery packs, AC/DC power adapters. These are eliminated because of the common use of USB ports in general electrical equipment, such as mobile phones, computers, and medical electrical equipment
Eliminates need for supporting brackets.
Eliminates need for country-specific power adapters.
Can be used in remote areas where power is not available, for example, using solar power, hand crank battery pack
Fewer components.
Low power consumption.
Plug and play operation
Can communicate with a host controller, with ability to collect patient dosing data and communicate it to the host.
Avoidance of need to integrate a control PCB into medical electrical equipment in order to operate from the ME GUI (graphical user interface). For example, it is known at present to integrate an Aerogen™ control PCB into a ventilator, to enable operation of nebulizers via the ventilator interface. The present invention will remove the need for such integration.
Compatibility/usability with wide variety of medical electrical equipment (i.e. any equipment with USB port).
Can be powered by any of a range of devices such as USB battery packs or portable solar panels.

The controller may be hand-held having a battery and power supply akin to that of a mobile phone.

The invention is not limited to the embodiments described but may be varied in construction and detail.

1-12. (canceled)

13. A power and communications connection device, comprising:
a first cable including power and data channels and having a first plug optionally connectable to either a host computing device or a ventilator, the ventilator being separate and distinct from both the host computing device and the power and communications connection device;
a second cable including power and data channels and having a second plug connectable to a nebulizer to operate the nebulizer with either the host computing device or the ventilator; and
a controller disposed between the first cable and the second cable, the controller configured to:
when the first plug is connected to the host computing device, operate the nebulizer according to a first mode determined by the host computing device; and
when the first plug connected to the ventilator, operate the nebulizer according to a second mode determined by a program stored on the controller.

14. The power and communications connection device of claim 13, wherein the controller includes a housing having a user interface.

15. The power and communications connection device of claim 14, wherein, when the first plug is connected to the host computing device, the controller is further configured to limit local control of the controller via the user interface to on/off control.

16. The power and communications connection device of claim 13, wherein the second mode is a patient-specific dosing regime.

17. The power and communications connection device of claim 16, wherein the patient-specific dosing regime is downloaded to the controller from an external source.

18. The power and communications connection device of claim 13, wherein, when the first plug is connected to the host computing device, the controller is further configured to send information to the host computing device, the information including one or more of a wet/dry state of the nebulizer or a nebulizer duration and time of nebulization.

19. The power and communications connection device of claim 13, wherein the host computing device includes medical equipment, a laptop computer, or a desktop computer.

20. The power and communications connection device of claim 13, wherein the first plug is a USB plug.

21. The power and communications connection device of claim 13, wherein the first plug and the second plug are different types of plugs.

22. The power and communications connection device of claim 13, wherein, when the first plug is connected to the ventilator, the controller is further configured to receive power from the ventilator and provide power to the nebulizer.

23. A power and communications connection device, comprising:
a first cable including power and data channels and having a first plug connectable to a power supplying device;
a second cable including power and data channels and having a second plug connectable to a nebulizer; and
a controller disposed between the first cable and the second cable, the controller configured to:
download a patient-specific dosing regime from an external source; and
operate the nebulizer according to the patient-specific dosing regime.

24. The power and communications connection device of claim 23, wherein the controller includes a user interface and wherein the controller is further configured to operate the nebulizer according to the patient-specific dosing regime based at least in part on input received through the user interface.

25. The power and communications connection device of claim 24, wherein the controller further includes a housing and wherein the user interface is included in the housing.

26. The power and communications connection device of claim 23, wherein the power supplying device is a ventilator.

27. The power and communications connection device of claim 23, wherein the controller is configured to receive power from the power supplying device and provide power to the nebulizer.

28. A method of controlling a nebulizer, comprising:
receiving, at a controller, power from a power supplying device via a first cable extending from a first end of the controller;
providing, by the controller, power to a nebulizer via a second cable extending from a second end of the controller; and
operating the nebulizer according to a patient-specific dosing regime stored on the controller.

29. The method of claim 28, further comprising receiving the patient-specific dosing regime from an external source.

30. The method of claim 28, further comprising:
receiving input through a user interface included in the controller; and
operating the nebulizer according to the patient-specific dosing regime based at least in part on the input received through the user interface.

31. The method of claim 28, wherein the power supplying device is a ventilator.

32. The method of claim 28, further comprising:
connecting the controller to a host computing device via the first cable; sending, by the controller, information to the host computing device, the information including one or more of a wet/dry state of the nebulizer or a nebulizer duration and time of nebulization.
